(19) United States
(12) Patent Application Publication
(10) Pub. No.: US 2005/0215879 A1
(43) Pub. Date: Sep. 29, 2005

(54) Accuracy evaluation of video-based augmented reality enhanced surgical navigation systems

(75) Inventor: Zhu Chuanggui, Singapore (SG)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco Imaging, S.p.A., Milano (IT)

(21) Appl. No.: 11/080,172

(22) Filed: Mar. 14, 2005

Related U.S. Application Data
(60) Provisional application No. 60/552,565, filed on Mar. 12, 2004.

(51) Int. Cl. A61B 5/05

(52) U.S. Cl. 600/407

(57) ABSTRACT

Systems and methods for measuring overlay error in a video-based augmented reality enhanced surgical navigation system are presented. In exemplary embodiments of the present invention the system and method include providing a test object, creating a virtual object which is a computer model of the test object, registering the test object, capturing images of control points on the test object at various positions within an augmented reality system's measurement space, and extracting positions of control points on the test object from the captured images, calculating the positions of the control points in virtual image, and calculating the positional difference of positions of corresponding control points between the respective video and virtual images of the test object. The method and system can further assess if the overlay accuracy meets an acceptable standard. In exemplary embodiments of the present invention a method and system are provided to identify the various sources of error in such systems and assess their effects on system accuracy. In exemplary embodiments of the present invention, after the accuracy of an AR system is determined, the AR system may be used as a tool to evaluate the accuracy of other processes in a given application, such as registration error.

### CROSS-REFERENCE TO RELATED APPLICATIONS

This application claims the benefit of United States Provisional Patent Application No. 60/552,565, filed on Mar. 12, 2004, which is incorporated herein by this reference. This application also claims priority to U.S. Utility patent application Ser. No. 10/832,902 filed on Apr. 27, 2004 (the “Camera Probe Application”).

### FIELD OF THE INVENTION

The present invention relates to video-based augmented reality enhanced surgical navigation systems, and more particularly to methods and systems for evaluating the accuracy of such systems.

### BACKGROUND OF THE INVENTION

Image guidance systems are increasingly being used in surgical procedures. Such systems have been proven to increase the accuracy and reduce the invasiveness of a wide range of surgical procedures. Currently, image guided surgical systems (“Surgical Navigation Systems”) are based on obtaining a pre-operative series of scan or imaging data, such as, for example, Magnetic Resonance Imaging (“MRI”), Computerized Tomography (“CT”), etc., which can then be registered to a patient in the physical world by various means.

In many conventional image guided operations, volumetric data, or three dimensional (“3D”) data, created from pre-operative scan images is displayed as two dimensional images in three orthogonal planes which change according to the three dimensional position of the tip of a tracked probe holding by a surgeon. When such a probe is introduced into a surgical field, the position of its tip is generally represented as an icon drawn on such images, so practitioners actually see a moving icon in each of three 2D views.¹ By linking preoperatively obtained imaging data with an actual surgical field (i.e., a real-world perceptible human body in a given 3D physical space), navigation systems can provide a surgeon or other practitioner with valuable information not immediately visible to him within the surgical field. For example, such a navigation system can calculate and display the exact localization of a currently held tool in relation to surrounding structures within a patient's body. In an AR system such as is described in the Camera Probe Application, the surrounding structures can be part of the scan image. They are aligned with a patient's corresponding real structures through the registration process. Thus, what can be seen on the monitor is the analogous point of the held probe (its position difference to the real tip is the tracking error) in relationship to the patient's anatomic structure in the scan image (the position difference of a point on the anatomic structure to its equivalent on the patient is the registration error at that point). This can help to relate actual tissues of an operative field to the images (of those tissues and their surrounding structures) used in pre-operative planning.
¹ The views presented are commonly the axial, coronal and saggital slices through the area of interest.

There is an inherent deficiency in such a method. Because in such conventional systems the displayed images are only two dimensional, to be fully utilized they must be mentally reconciled into a three dimensional image by a surgeon (or other user) as he works. Thus, sharing a problem which is common to all conventional navigation systems which present pre-operative imaging data in 2D orthogonal slices, a surgeon has to make a significant mental effort to relate the spatial information in a pre-operative image series to the physical orientation of the patient's area of interest. Thus, for example, a neurosurgeon must commonly relate a patient's actual head (which is often mostly covered by draping during an operation) and the various structures within it to the separate axial, saggital and coronal image slices obtained from pre-operative scans.

Addressing this problem, some conventional systems display a three dimensional (“3D”) data set in a fourth display window. However, in such systems the displayed 3D view is merely a 3D rendering of pre-operative scan data and is not at all correlated to, let alone merged with, a surgeon's actual view of the surgical field. As a result a surgeon using such systems is still forced to mentally reconcile the displayed 3D view with his real time view of the actual field. This often results in a surgeon continually switching his view between the 3D rendering of the object of interest (usually presented as an “abstract” object against a black background) and the actual real world object he is working on or near.

To overcome these shortcomings, Augmented Reality (AR) can be used to enhance image guided surgery. Augmented Reality generates an environment in which computer generated graphics of virtual objects can be merged with a user's view of real objects in the real world. This can be done, for example, by merging a 3D rendering of virtual objects with a real time video signal obtained from a video-camera (video-based AR), projecting the virtual objects into a Head Mounted Display (HMD) device, or even projecting such virtual objects directly onto a user's retina.

A video-based AR enhanced surgical navigation system generally uses a video camera to provide real-time images of a patient and a computer to generate images of virtual structures from the patient's three-dimensional image data obtained via pre-operative scans. The computer generated images are superimposed over the live video, providing an augmented display which can be used for surgical navigation. To make the computer generated images coincide precisely with their real equivalents in the real-time video image, (i) virtual structures can be registered with the patient and (ii) the position and orientation of the video camera in relation to the patient can be input to the computer. After registration, a patient's geometric relationship to a reference system can be determined. Such a reference system can be, for example, a co-ordinate system attached to a 3D tracking device or a reference system rigidly linked to the patient. The camera-to-patient relationship can thus be determined by a 3D tracking device which couples to both the patient as well as to the video camera.

Just such a surgical navigation system is described in the copending Camera Probe Application. The system therein described includes a micro camera in a hand-held navigation probe which can be tracked by a tracking system. This enables navigation within a given operative field by viewing real-time images acquired by the micro-camera that are combined with computer generated 3D virtual objects from prior scan data depicting structures of interest. By varying the transparency settings of the real-time images and the superimposed 3D graphics, the system can enhance a user's depth perception. Additionally, distances between the probe and superimposed 3D virtual objects can be dynamically displayed in or near the combined image. Using the Camera Probe technology, virtual reality systems can be used to plan surgical approaches using multi-modal CT and MRI data acquired pre-operatively, and the subsequent transfer of a surgical planning scenario into real-time images of an actual surgical field is enabled.

Overlay of Virtual and Real Structures; Overlay Error

In such surgical navigation systems, it is crucial that the superimposed images of virtual structures (i.e., those generated from a patent's pre-operative volumetric data) coincide precisely with their real equivalents in the real-time combined image. Various sources of error, including registration error, calibration error, and geometric error in the volumetric data, can introduce inaccuracies in the displayed position of certain areas of the superimposed image relative to the real image. As a result, when a 3D rendering of a patient's volumetric data is overlaid on a real-time camera image of that patient, certain areas or structures appearing in the 3D rendering may be located at a slightly different place than the corresponding area or structure in the real-time image of the patient. Thus, a surgical instrument that is being guided with reference to locations in the 3D rendering may not be directed exactly to the desired corresponding location in the real surgical field.

General details on the various types of error arising in surgical navigation systems are discussed in William Hoff and Tyrone Vincent, Analysis of Head Pose Accuracy in Augmented Reality. IEEE Transactions on Visualization and Computer Graphics, vol. 6, No. 4, October-December 2000.

For ease of description herein, error in the positioning of virtual structures relative to their real equivalents in an augmented image shall be referred to as “overlay error.” For an augmented reality enhanced surgical navigation system to provide accurate navigation and guidance information, the overlay error should be limited to be within an acceptable standard. ²
² An example of such an acceptable standard can be, for example, a two pixels standard deviation of overlay errors between virtual structures and their real-world equivalents in the augmented image across the whole working space of an AR system under ideal application conditions. “Ideal application conditions,” as used herein, can refer to (i) system configurations and set up being the same as in the evaluation; (ii) no errors caused by applications such as modeling errors and tissue deformation are present; and (iii) registration error is as small as in the evaluation.

Visual Inspection

One conventional method of overlay accuracy evaluation is visual inspection. In such a method a simple object, such as a box or cube, is modeled and rendered. In some cases, a mockup of a human head with landmarks is scanned by means of CT or MRI, and virtual landmarks with their 3D coordinates in the 3D data space are used instead. The rendered image is then superimposed on a real-time image of the real object. The overlay accuracy is evaluated by examining the overlay error from different camera positions and angles. To show how accurate the system is, usually several images or a short video are recorded as evidence.

A disadvantage of this approach is that a simple visual inspection does not provide a quantitative assessment. Though this can be amended by measuring the overlay error between common features of virtual and real objects in the augmented image by measuring the positional difference between a feature on a real object and the corresponding feature on a virtual object in a combined AR image, the usefulness of such a measurement often suffers due to (1) the number of features are usually limited; (2) the chosen features only sample a limited portion of the working space; and (3) the lack of accuracy in modeling, registration and location of the features.

A further disadvantage is that such an approach fails to separate overlay errors generated by the AR system from errors introduced in the evaluation process. Potential sources of overlay inaccuracy can include, for example, CT or MRI imaging errors, virtual structure modeling errors, feature locating errors, errors introduced in the registration of the real and virtual objects, calibration errors, and tracking inaccuracy. Moreover, because some error sources, such as those associated with virtual structure modeling and feature location are not caused by the AR system their contribution to the overlay error in an evaluation should be removed or effectively suppressed.

Furthermore, this approach does not distinguish the effects of the various sources of error, and thus provides few clues for the improvement of system accuracy.

Numerical Simulation

Another conventional approach to the evaluation of overlay accuracy is the “numerical simulation” method. This method seeks to estimate the effects of the various error sources on overlay accuracy by breaking the error sources into different categories, such as, for example, calibration errors, tracking errors and registration errors. Such a simulation generally uses a set of target points randomly generated within a pre-operative image. Typical registration, tracking and calibration matrices, normally determined by an evaluator from an experimental dataset, can be used to transform these points from pre-operative image coordinates to overlay coordinates. (Details on such matrices are provided in Hoff and Vincent, supra). The positions of these points in these different coordinate spaces are often used as an error-free baseline or “gold standard.” A new set of slightly different registration, tracking and calibration matrices can then be calculated by including errors in the determination of these matrices. The errors can be randomly determined according to their Standard Deviation (SD) estimated from the experiment dataset. For example, the SD of localization error in the registration process could be 0.2 mm. The target points are transformed again using this new set of transform matrices. The position differences of the target points to the ‘gold standard’ in different coordinate space are the errors at various stages. This process can be iterated a large number of times, for example 1000 times, to get a simulation result.

There are numerous problems with numerical simulation. First, the value of SD error is hard to determine. For some error sources it may be too difficult to obtain an SD value and thus these sources cannot be included in the simulation. Second, the errors may not be normally distributed and thus the simulation may not be accurate. Third, simulation needs real measurement data to verify the simulation result. Thus, without verification, it is hard to demonstrate that a simulation can mimic a real-world scenario with any degree of confidence. Finally—but most importantly—such a simulation cannot tell how accurate a given individual AR system is because the simulation result is a statistical number which generally gives a probability as to the accuracy of such a system by type (for example, that 95% of such systems will be more accurate than 0.5 mm). In reality, each actual system of a given type or kind should be evaluated to prove that its error is below a certain standard, for example SD 0.5 mm, so that if it is not, the system can be recalibrated, or even modified, until it does meet the standard.

What is thus needed in the art is an evaluation process that can quantitatively assess the overlay accuracy of a given AR enhanced surgical navigation system, and that can further assess if that overlay accuracy meets an acceptable standard. Moreover, such a system should evaluate and quantify the individual contributions to the overall overlay accuracy by the various sources of error.

### SUMMARY OF THE INVENTION

Systems and methods for measuring overlay error in a video-based augmented reality enhanced surgical navigation system are presented. In exemplary embodiments of the present invention the system and method include providing a test object, creating a virtual object which is a computer model of the test object, registering the test object, capturing images of control points on the test object at various positions within an augmented reality system's measurement space, and extracting positions of control points on the test object from the captured images, calculating the positions of the control points in virtual image, and calculating the positional difference of positions of corresponding control points between the respective video and virtual images of the test object. The method and system can further assess if the overlay accuracy meets an acceptable standard. In exemplary embodiments of the present invention a method and system are provided to identify the various sources of error in such systems and assess their effects on system accuracy. In exemplary embodiments of the present invention, after the accuracy of an AR system is determined, the AR system may be used as a tool to evaluate the accuracy of other processes in a given application, such as, for example, registration error.

### BRIEF DESCRIPTION OF THE DRAWINGS

FIG. 1 is a process flow diagram of an exemplary method of accuracy assessment according to an exemplary embodiment of the present invention;

FIG. 2 illustrates the definition of image plane error (IPE) and object space error (OSE) as used in exemplary embodiments of the present invention;

FIG. 3 depicts an exemplary bi-planar test object according to an exemplary embodiment of the present invention;

FIG. 4 depicts a virtual counterpart to the test object of FIG. 3 according to an exemplary embodiment of the present invention;

FIG. 5 illustrates a defined accuracy space according to an exemplary embodiment of the present invention;

FIG. 6 depicts an exemplary registration process flow according to an exemplary embodiment of the present invention;

FIG. 7 is an exemplary screen shot indicating registration errors resulting from a fiducial based registration process according to an exemplary embodiment of the present invention;

FIGS. 8(a)-(b) illustrate the use of an AR system whose accuracy has been determined as an evaluation tool to assess the registration error of an object according to an exemplary embodiment of the present invention;

FIGS. 9(a)-(b) illustrate the use of an AR system whose accuracy has been determined as an evaluation tool to assess the registration error of internal target objects according to an exemplary embodiment of the present invention;

FIG. 10 depicts 27 exemplary points used for registration of an exemplary test object according to an exemplary embodiment of the present invention;

FIGS. 11 (a)-(c) are snapshots from various different camera positions of an exemplary overlay display for an exemplary planar test object which was used to evaluate an AR system according to an exemplary embodiment of the present invention;

FIG. 12 depicts an exemplary planar test object with nine control points indicated according to an exemplary embodiment of the present invention; and

FIG. 13 depicts an exemplary evaluation system using the exemplary planar test object of FIG. 12 according to an exemplary embodiment of the present invention.

It is noted that the patent or application file contains at least one drawing executed in color. Copies of this patent or patent application publication with color drawings will be provided by the U.S. Patent Office upon request and payment of the necessary fee.

### DETAILED DESCRIPTION OF THE INVENTION

In exemplary embodiments of the present invention systems and methods for assessing the overlay accuracy of an AR enhanced surgical navigation system are provided. In exemplary embodiments of the present invention the method can additionally be used to determine if the overlay accuracy of a given AR system meets a defined standard or specification.

In exemplary embodiments of the present invention methods and corresponding apparatus can facilitate the assessment of the effects of various individual error sources on overall accuracy, for the purpose of optimizing an AR system.

Using the methods of the present invention, once the overlay accuracy of a given AR system has been established, that AR system can itself be used as an evaluation tool to evaluate the accuracy of other processes which can affect overlay accuracy in a given application, such as, for example, registration of prior scan data to a patient.

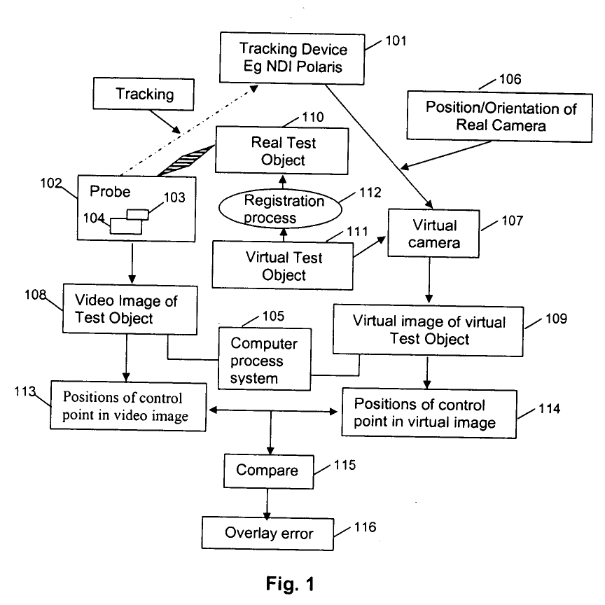

FIG. 1 illustrates an exemplary overlay accuracy evaluation process according to an exemplary embodiment of the present invention. The process can be used, for example, to evaluate a given AR enhanced surgical navigation system, such as, for example, that described in the Camera Probe Application.

With reference to FIG. 1, an exemplary AR system to be evaluated comprises an optical tracking device 101, a tracked probe 102 and a computer 105 or other data processing system. The probe contains an attached reference frame 103 and a micro video camera 104. The reference frame 103 can be, for example, a set of three reflective balls detectable by a tracking device, as described in the Camera Probe Application. These three balls, or similar marker apparatus as is known in the art, can thus determine a reference frame attached to the probe.

The tracking device can be, for example, optical, such as, for example, an NDI Polaris™ system, or any other acceptable tracking system. Thus, the 3D position and orientation of the probe's reference frame in the tracking device's coordinate system can be determined. It is assumed herein that the exemplary AR system has been properly calibrated and that the calibration result has been entered into computer 105. Such a calibration result generally includes the intrinsic parameters of the AR system camera, such as, for example, camera focal length fx and fy, image center Cx and Cy, and distortion parameters k(1), k(2), K(3) and k(4), as well as a transform matrix from the camera to the probe's reference frame,

$${TM}_{cr} = [ \begin{matrix}{R}_{cr} & 0 \\ {T}_{cr} & 1\end{matrix} ] .$$

In this transform matrix R_{cr} refers to the orientation of the camera within the coordinate system of the probe's reference frame, while T_{cr} refers to the position of the camera within the coordinate system of the probe's reference frame. The matrix thus provides the position and orientation of the camera 106 within the probe's reference frame. Using these parameters a virtual camera 107 can, for example, be constructed and stored in computer 105.

Such an example AR surgical navigation system can mix, in real-time, real-time video images of a patient acquired by a micro-camera 104 in the probe 102 with computer generated virtual images generated from the patient's pre-operative imaging data stored in the computer 105. To insure that the virtual structures in the virtual images coincide with their real-world equivalents as seen in the real-time video, the pre-operative imaging data can be registered to the patient and the position and orientation of the video camera in relation to the patient can be updated in real time by, for example, tracking the probe.

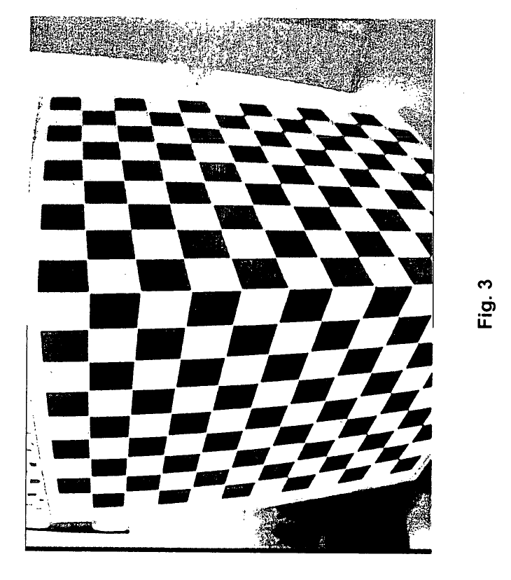

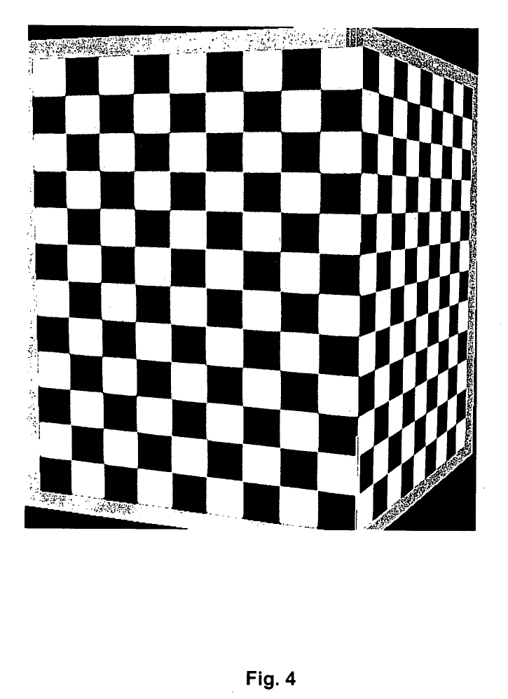

In exemplary embodiments of the present invention, a test object 110 can be used, for example, to evaluate the overlay accuracy of an exemplary AR surgical navigation system as described above. (It is noted that a test object will sometimes be referred to herein as a “real test object” to clearly distinguish from a “virtual test object”, as for example, in 110 of FIG. 1). The test object can be, for example, a three-dimensional object with a large number of control, or reference, points. A control point is a point on the test object whose 3D location within a coordinate system associated with the test object can be precisely determined, and whose 2D location in an image of the test object captured by the video camera can also be precisely determined. For example, the corners of the black and white squares can be used as exemplary control points on the exemplary test object of FIG. 3. In order to accurately test the overlay accuracy of a given AR system over a given measurement volume, control points can, for example, be distributed throughout it. Additionally, in exemplary embodiments of the present invention, control points need to be visible in an image of the test object acquired by the camera of the AR system under evaluation, and their positions in the image easily identified and precisely located.

In exemplary embodiments of the present invention, a virtual test object 111 can, for example, be created to evaluate the overlay accuracy of an exemplary AR surgical system such as is described above. A virtual image 109 of the virtual test object 111 can be generated using a virtual camera 107 of the AR system in the same way as the AR system renders other virtual structures in a given application. A virtual camera 107 mimics the imaging process of a real camera. It is a computer model of a real camera, described by a group of parameters obtained, for example, through the calibration process, as described above. A “virtual test object” 111 is also a computer model which can be imaged by the virtual camera, and the output is a “virtual image” 109 of virtual object 111. For clarity of the following discussion, a computer generated image shall be referred to herein as a “virtual image”, and an image (generally “real time”) from a video camera as a “video image.” In exemplary embodiments of the present invention, the same number of control points as are on the real test object 110 are on the virtual test object 111. The control points on the virtual test object 111 can be seen in the virtual image 109 generated by the computer. Their positions in the image can be easily identified and precisely located.

As noted above, a virtual test object 111 is a computer generated model of a real test object 110. It can, for example, be generated using measurements taken from the test object. Or, for example, it can be a model from a CAD design and the physical test object can be made from this CAD model. Essentially, in exemplary embodiments of present invention the test object and the corresponding virtual test object should be geometrically identical. In particular, the control points on each of the test object and the virtual test object must be geometrically identical. While identity of the other parts of the test object to those of the virtual test object is preferred, it is not a necessity.

It is noted that the process of creating a virtual test object can introduce a modeling error. However, this modeling error can be controlled to be less than 0.01 mm with current technology (it being noted that using current technology it is possible to measure and manufacture to tolerances as small as 10⁻⁷ m, such as, for example, in the semi-conductor chip making industry) which is much more accurate than the general range of state of the art AR system overlay accuracy. Thus, the modeling error can generally be ignored in exemplary embodiments of the present invention.

In exemplary embodiments of the present invention, a virtual test object 111 can be registered to a corresponding real test object 110 at the beginning of an evaluation through a registration process 112. To accomplish such registration, as, for example, in the exemplary AR system of the Camera Probe Application, a 3D probe can be tracked by a tracking device and used to point at control points on the test object one by one while the 3D location of each such point in the tracking device's coordinate system is recorded. In exemplary embodiments of the present invention such a 3D probe can, for example, be a specially designed and precisely calibrated probe so that the pointing accuracy is higher than a standard 3D probe as normally used in an AR application, such as, for example, surgical navigation as described in the Camera Probe Application.

For example, such a special probe can (1) have a tip with an optimized shape so that it can touch a control point on a test object more precisely; (2) have its tip's co-ordinates within the reference frame of the probe determined precisely using a calibration device; and/or (3) have an attached reference frame comprising more than three markers, distributed in more than one plane, with larger distances between the markers. The markers can be any markers, passive or active, which can be tracked most accurately by the tracking device. Thus, using such a specialized probe, control points on the real test object can be precisely located with the probe tip. This allows for a precise determination of their respective 3D coordinates in the tracking device's coordinate system. At a minimum, in exemplary embodiments of the present invention, the 3D locations of at least three control points on a test object can be collected for registration. However, in alternate exemplary embodiments, many more control points, such as, for example, 20 to 30, can be used so that the registration accuracy can be improved by using an optimization method such as, for example, a least square method.

To reduce pointing error and thus further improve registration accuracy, a number of pivots³, for example, can be made when the real test object is manufactured. Such pivots can, for example, be precisely aligned with part of the control points, or, if they are not precisely aligned, their positions relative to the control points can be precisely measured. A pivot can, for example, be designed in a special shape so that it can be precisely aligned with the tip of a probe. In exemplary embodiments of the present invention, at least three such pivots can be made on the test object, but many more can alternatively be used to improve registration accuracy, as noted above. When using pivots, registration can be done, for example, by pointing at the pivots instead of pointing at the control points.
³ A pivot is a cone shaped pit to trap the tip of a 3D probe to a certain position in regardless of the probes rotation. To make the pointing even more accurate, the shape of the pivot could be made matching the shape of the probe tip.

After registration, a virtual test object can be, for example, aligned with the real test object and the geometric relationship of the real test object to the tracking device can be determined. This geometric relationship can, for example, be represented as a transform matrix

$${TM}_{ot} = [ \begin{matrix}{R}_{ot} & 0 \\ {T}_{ot} & 1\end{matrix} ] .$$

In this matrix Rₒₜ refers to the orientation of the test object within the coordinate system of the tracking device, while Tₒₜ refers to the position of the test object within the coordinate system of the tracking device.

The probe 102 can, for example, be held at a position relative to the tracking device 101 where it can be properly tracked. A video image 108 of the test object 110 can be captured by the video camera. At the same time the tracking data of the reference frame on the probe can be recorded and the transform matrix from the reference frame to the tracking device, i.e.,

$${TM}_{rt} = [ \begin{matrix}{R}_{rt} & 0 \\ {T}_{rt} & 1\end{matrix} ] ,$$

can be determined. In this expression Rᵣₜ refers to the orientation of the probe's reference frame within the coordinate system of the tracking device, and Tᵣₜ refers to the position of the probe's reference frame within the coordinate system of the tracking device.

Then, in exemplary embodiments of the present invention, the transform matrix from the camera to the real test object TM_{co} can be calculated from the tracking data, registration data, and calibration result using the formula TM_{co}=TM_{cr}·TMᵣₜ·TMₒₜ⁻¹, where TM_{co} contains the orientation and position of the camera to the test object. Using the value of TM_{co}, the stored data of the virtual camera (i.e., the calibration parameters as described above), and the virtual test object, the computer can, for example, generate a virtual image 109 of the virtual test object in the same way, for example, as is done in an application such as surgical navigation as described in the Camera Probe Application.

The 2D locations of control points 113 in video image 108 can be extracted using methods known in the art, such as, for example, for corners as control points, Harrie's corner finder method, or other corner finder methods as are known in the art. The 3D position (Xₒ, Yₒ, Zₒ) of a control point in the test object coordinate system can be known from either manufacturing or measurement of the test object. Its 3D position (X_{c}, Y_{c}, Z_{c}) in relation to the camera can be obtained by the expression (X_{c} Y_{c} Z_{c})=(Xₒ Yₒ Zₒ)·TM_{co}. Thus, in exemplary embodiments of the present invention, the 2D locations of control points 114 in the virtual image 109 can be given directly by computer 105.

Finding the correspondence of a given control point in video image 108 to its counterpart in corresponding virtual image 109 does not normally present a problem inasmuch as the distance between the corresponding points in the overlay image is much smaller than the distance to any other points. Moreover, even if the overlay error is large, the corresponding control point problem can still be easily solved by, for example, comparing features in the video and virtual images.

Continuing with reference to FIG. 1, at 115 the 2D locations of control points in the video image can be, for example, compared with the 2D locations of their corresponding points in the virtual image in a comparing process 115. The locational differences between each pair of control points in video image 108 and virtual image 109 can thus be calculated.

The overlay error can be defined as the 2D locational differences between the control points in video image 108 and virtual image 109. For clarity of the following discussion, such overlay error shall be referred to herein as Image Plane Error (IPE). For an individual control point, the IPE can be defined as:
IPE=\sqrt{(Δx)²+(Δy)²},
where Δx and Δy are the locational differences for that control point's position in the X and Y directions between the video 108 and virtual 109 images.

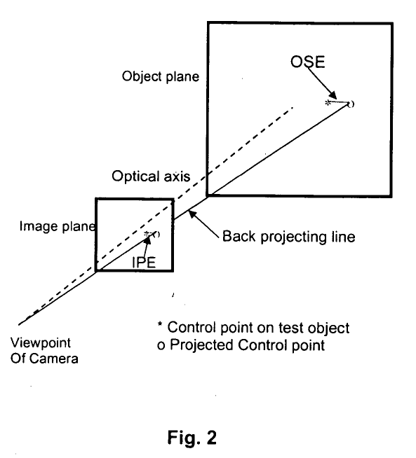

The IPE can be mapped into 3D Object Space Error (OSE). There can be different definitions for OSE. For example, OSE can be defined as the smallest distance between a control point on the test object and the line of sight formed by back projecting through the image of the corresponding control point in virtual image. For simplicity, the term OSE shall be used herein to refer to the distance between a control point and the intersection point of the above-mentioned line of sight with the object plane. The object plane is defined as the plane that passes through the control point on the test object and parallels with the image plane, as is illustrated in FIG. 2.

For an individual control point the OSE can be defined as:
OSE=\sqrt{(ΔxZ_{c}/fx)²+(ΔyZ_{c}/fy)²},
where fx and fy are the effective focal length of the video camera in X and Y directions, known from the camera calibration. Zc is the distance from the viewpoint of the video camera to the object plane, and Δx and Δy are the locational differences of the control point in the X and Y directions in the video and virtual images, defined in the same manner as for the IPE.

An AR surgical navigation system's overlay accuracy can thus be determined by statistical analysis of the IPE and OSE errors calculated from the location differences of corresponding control points in video image and virtual image, using the methods of an exemplary embodiment of this invention. The overlay accuracy can be reported in various ways as are known in the art, such as, for example, maximum, mean, and root-mean-square (RMS) values of IPE and OSE. For an exemplary AR system (a version of the DEX-Ray system described in the Camera Probe Application) which was evaluated by the inventor, the maximum, mean and RMS IPE were 2.24312, 0.91301, and 0.34665 respectively, in units of pixels, and the corresponding maximum, mean and RMS OSE values were 0.36267, 0.21581, and 0.05095 in mm. This is about ten times better than the application error of current IGS systems for neurosurgery. It is noted that this result represents the system accuracy. In any given application using the evaluated system, the overall application error may be higher due to other error sources inherent in such application.

In exemplary embodiments of the present invention, a virtual test object can be, for example, a data set containing the control points' 3D locations relative to the coordinate system of the test object. A virtual image of a virtual test object can, for example, consist of the virtual control points only. Or, alternatively, the virtual control points can be displayed using some graphic indicator, such as a cross hair, avatar, asterisk, etc. Or, alternatively still, the virtual control points can be “projected” onto the video images using graphics. Or, even alternatively, for example, their positions need not be displayed at all, as in any event their positions are calculated by the computer, as the virtual image is generated by the computer, so the computer already “knows” the attributes of the virtual image, including the locations of its virtual control points .

In exemplary embodiments of the present invention, a (real) test object can, for example, be a bi-planar test object as is illustrated in FIG. 3. This exemplary test object comprises two connected planes with a checkerboard design. The planes are at right angles to one another (hence “bi-planar”). The test object's control points can be, for example, precisely manufactured or precisely measured, and thus the 3D locations of the control points can be known to a certain precision.

In exemplary embodiments of the present invention, a virtual test object can be, for example, created from the properties of the bi-planar test object as is shown in FIG. 4. Such a virtual test object is a computer model of the bi-planar test object. It can, for example, be generated from the measured data of the bi-planar test object and thus the 3D locations of the control points can be known to a pre-defined coordinate system of the bi-planar test object. The control points on both the test object and the virtual test object are identical geometrically. Thus, they have the same interpoint distances, and the same respective distances to the test object boundaries.

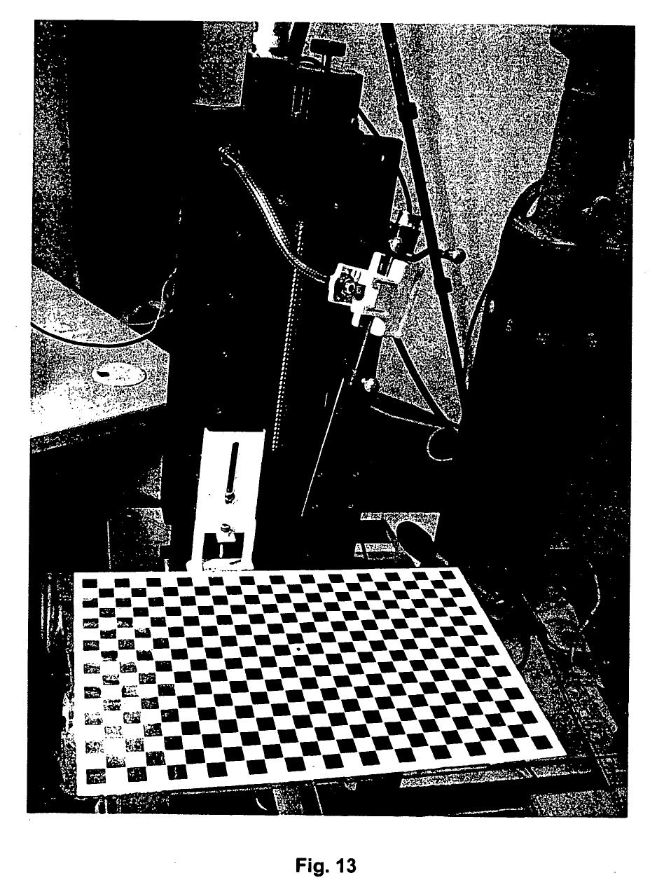

In exemplary embodiments of the present invention, a test object can consist of control points on a single plane. In such case, the test object can, for example, be stepped through a measurement volume by a precise moving device such as, for example, a linear moving stage. This evaluation apparatus is shown, for example, in FIG. 13. Accuracy evaluation can, for example, be conducted on, for example, a plane-by-plane basis in the same manner as has been described for a volumetric test object (i.e., the exemplary bi-planar test object of FIG. 3). A large number of points across the measurement volume can be reached through the movement of a planar test object and the coordinates of these points can be determined relative to the moving device by various means as are known in the art. The coordinates of these points relative to an optical, or other, tracking device can then be determined through a registration process similar to that described above in using a volumetric test object, i.e., by using a 3D probe to detect the control points' respective 3D positions at a certain number of different locations. In such case, the 3D probe can be held at a proper position detectable by the tracking device (as shown, for example, in FIG. 13). After registration, the control points' coordinates relative to the video camera can, for example, be determined in the same way as described above for a volumetric test object. The geometrical relationship of the control points at each given step can be determined by the registration result, the tracking data, and the AR system calibration data stored in the computer, in the same way as described above for a volumetric test object. Thus, a virtual image of the control points at each step can, for example, be generated by the computer. Also, a video image can, for example, be captured at each step and the overlay accuracy can then be determined at that step by calculating the locational differences between the control points in the video image and the same control points in the corresponding virtual image.

In exemplary embodiments of the present invention, a test object may even consist of a single control point. In such case, the test object can, for example, be stepped throughout the measurement volume by a precise moving device such as a coordinate measurement machine (CMM), such as, for example, the Delta 34.06 by DEA Inc., which has a volumetric accuracy of 0.0225 mm. Accuracy evaluation can be conducted, for example, for point-by-point bases using the same principles as described above for using a volumetric test object. A large number of points throughout the measurement volume can be reached by the movement of the test object and their respective coordinates to the moving device can be determined by various means as are known in the art. Their coordinates relative to a tracking device can then, for example, be determined through a registration process similar to that described above for a volumetric test object, i.e., by using a 3D probe to detect the control point's 3D position at a certain number of different locations. In such case, the probe can, for example, be held at a proper position which is detectable by the tracking device. After registration, the control point's coordinates to the video camera can be determined in the same way as with a planar test object. The geometrical relationship of the control points at each step through a measurement volume can be determined by the registration result, the tracking data, and the AR system calibration data stored in the computer, in the same way as was described for a volumetric test object. Thus, a virtual image of the control points at each moving step can be generated by the computer. A video image can be, for example, captured at each step and the overlay accuracy can be determined at that step by calculating the locational difference between the control point in the video image and the control point in the corresponding virtual image.

In exemplary embodiments according to the present invention a method can be used to assess if the overlay accuracy of an AR system meets a defined acceptance standard.

The producer of an AR surgical navigation system usually defines such an acceptance standard. This acceptance standard, sometimes referred to as the “acceptance criteria”, is, in general, necessary to qualify a system for sale. In exemplary embodiments according to the present invention an exemplary acceptance standard can be stated, for example, as:

The OSE value across a pre-defined volume is <=0.5 mm, as determined using the evaluation methods of an exemplary embodiment of the present invention. This is sometimes known as “sub-millimeter accuracy.”

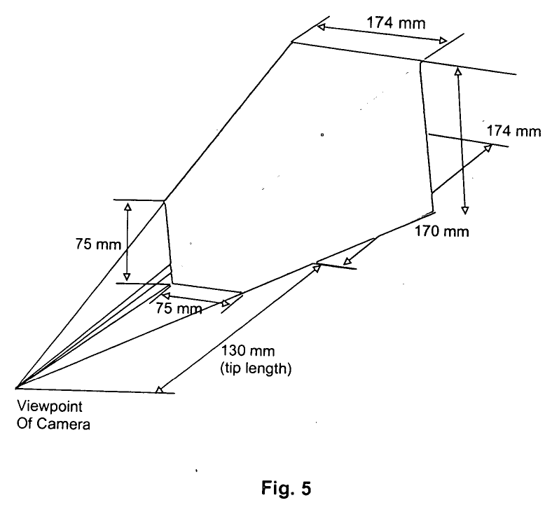

In exemplary embodiments according to the present invention the pre-defined volume can be referred to as the “accuracy space.” An exemplary accuracy space can be defined as a pyramidal space associated with a video camera, as is depicted in FIG. 5. The near plane of such exemplary accuracy space to the viewpoint of the camera is 130 mm. The depth of such pyramid is 170 mm. The height and width at the near plane are both 75 mm and at the far plane are both 174 mm, corresponding to a 512×512 pixel area in the image.

The overlay error may be different for different camera positions and orientations relative to the tracking device. This is because the tracking accuracy may depend on the position and orientation of the reference frame relative to the tracking device. The tracking accuracy due to orientation of the probe may be limited by the configurational design of the marker system (e.g., the three reflective balls on the DEX-Ray probe). As is known in the art, for most tracking systems it is preferred to have the plane of the reference frame perpendicular to the line of sight of the tracking system. However, the variety in tracking accuracy due to probe position changes can be controlled by the user. Thus, in exemplary embodiments of the present invention accuracy evaluation can be done at a preferred probe orientation because a user can achieve a similar probe orientation by adjusting the orientation of the probe to let the reference frame face the tracking device in an application. The overlay accuracy can also be visualized at the same time the overlay accuracy assessment is performed because the virtual image of the virtual control points can be overlaid on the video image of the real control points.

Thus the overlay accuracy at any probe position and orientation can be visually assessed in the AR display by moving the probe as it would be moved using an application.

In exemplary embodiments of the present invention an accuracy evaluation method and apparatus can be used to assess the effects of various individual error sources on overall accuracy, for the purpose of optimizing an AR system.

A test object as described above can be used to calibrate an AR system. After calibration, the same test object can be used to evaluate the overlay accuracy of such AR system. The effects on the overlay accuracy made by the contributions of different error sources, such as, for example, calibration and tracking, can be assessed independently.

As described above, the calibration of a video-based AR surgical navigation system includes calibration of the intrinsic parameters of the camera as well as calibration of the transform matrix from the camera to the reference frame on the probe. Camera calibration is well known in the art. Its function is to find the intrinsic parameters that describe the camera properties, such as focal length, image center and distortion, and the extrinsic parameters that are the camera position and orientation to the test object used for calibration. In the calibration process, the camera captures an image of a test object. The 2D positions of the control points in the image are extracted and their correspondence with the 3D positions of the control points to the test object are found. The intrinsic and extrinsic parameters of the camera can then be solved by a calibration program as is known in the art using the 3D and 2D positions of the control points as inputs.

An exemplary camera calibration for an exemplary camera from an AR system is presented below.

Intrinsic Parameters

Image Size: Nx=768, Ny=576
Focal Length: fx=885.447580, fy=888.067052
Image Center: Cx=416.042786, Cy=282.107896
Distortion: kc(1)=−0.440297, kc(2)=0.168759, kc(3)=−0.002408, kc(4)=−0.002668
Extrinsic Parameters

$$Tco = \begin{matrix}- 174.545851 & 9.128410 & - 159.505843\end{matrix}$$

$$Rco = \begin{matrix}0.635588 & 0.015614 & - 0.771871 \\ - 0.212701 & 0.964643 & - 0.155634 \\ 0.742150 & 0.263097 & 0.616436\end{matrix}$$

In exemplary embodiments of the present invention, as noted above, the transform matrix from the camera to the test object can be determined by calibration. Without tracking, a virtual image of the test object can be generated using the calibrated parameters. The virtual image can be compared with the video image used for calibration and the overlay error can be calculated. Because the overlay accuracy at this point only involves error introduced by the camera calibration, the overlay error thus can be used as an indicator of the effect of camera calibration on overall overlay error. In exemplary embodiments of the present invention this overlay accuracy can serve as a baseline or standard with which to assess the effect of other error sources by adding these other error sources one-by-one in the imaging process of the virtual image.

The transform matrix from the test object to the tracking device can be obtained by a registration process as described above. The transform matrix from the reference frame to the tracking device can be obtained directly through tracking inasmuch as the reference frame on the probe is defined by the marker, such as, for example, the three reflective balls, which are tracked by the tracking device. Thus the transform matrix from the camera to the reference frame can be calculated as
TM_{cr}=TM_{co}·TMₒₜ·TMᵣₜ⁻¹.

After calibration, the transform matrix from the camera to the test object can be obtained from tracking the reference frame. To evaluate the effects of tracking error on the overlay accuracy, the camera and the test object can be, for example, kept at the same positions as in calibration and the tracking device, and, for example, can be moved to various positions and orientations, preferably positioning the probe throughout the entire tracking volume of the tracking device. From the equation TM_{co}=TM_{cr}·TMᵣₜ·TMₒₜ⁻¹, it is clear that the effect of the tracking accuracy on the overlay error across the entire tracking volume, with different camera positions and orientations relative to the tracking device, can be assessed by recording a pair of images of the real and virtual calibration objects at each desired position and orientation, and then comparing the differences between the control points in each of the real and virtual images, respectively.

Using an Evaluated AR System as an Evaluation Tool

In exemplary embodiments according to the present invention, after the overlay accuracy has been assessed and proven to be accurate to within a certain standard, an AR system can then itself be used as a tool to evaluate other error sources which may affect the overlay accuracy.

For example, in exemplary embodiments according to the present invention, such an evaluated AR system (“EAR”) can, for example, be used to evaluate registration accuracy in an application.

There are many known registration methods used to align a patient's previous 3D image data with the patient. All of them rely on the use of common features in both the 3D image data and the patient. For example, fiducials, landmarks or surfaces are usually used for rigid object registration. Registration is a crucial step both for traditional image guided surgery as well as for AR enhanced surgical navigation. However, to achieve highly accurate registration is quite difficult, and to evaluate the registration accuracy is equally difficult.

However, using an AR system to assess the effect of registration errors is quite easy. Thus, in exemplary embodiments of the present invention, after registration, the overlay errors between features or landmarks appearing in both real and virtual images can be easily visualized, and any overlay errors exceeding the accuracy standard to which the AR system was evaluated can be assumed to have been caused by registration. Moreover, quantitative assessment is also possible by calculating the positional differences of these features in both real and virtual images.

In an exemplary embodiment according to the present invention, a phantom of a human skull with six fiducials was used by the inventor to demonstrate this principle. Four geometric objects in the shapes of a cone, a sphere, a cylinder, and a cube, respectively, were installed in the phantom as targets for registration accuracy evaluation. A CT scan of the phantom (containing the four target objects) was conducted. The surface of the phantom and the four geometric objects were segmented from the CT data.

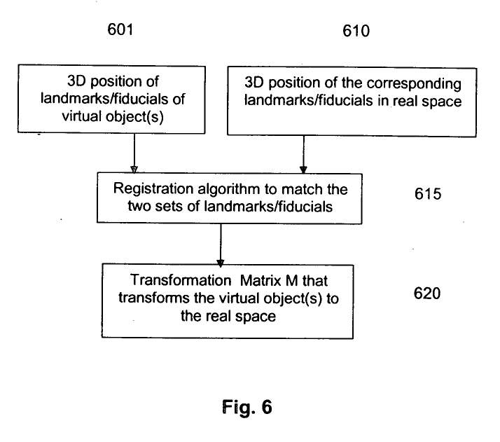

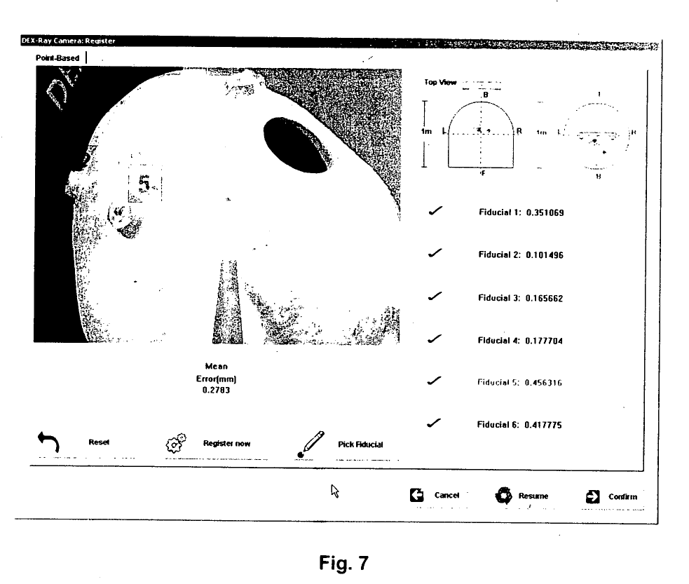

The fiducials in the CT scan data were identified and their 3D locations in the scan image coordinate system were recorded. Additionally, their 3D locations in the coordinate system of an optical tracking device were detected by pointing to them one by one with a tracked 3D probe, as described above. A known fiducial based registration process, as is illustrated in FIG. 6, was then conducted. At 601 the 3D positions of landmarks/fiducials of virtual objects were input, as were the 3D positions of the corresponding landmarks/fiducials in real space at 610, to a registration algorithm 615. The algorithm 615 generated a Transformation Matrix 620 that expresses the transformation of virtual objects to the real space. The registration errors from this process are depicted in FIG. 7, which is a screen shot of an exemplary interface of the DEX-Ray™ AR system provided by Volume Interactions Pte Ltd of Singapore, which was used to perform the test.

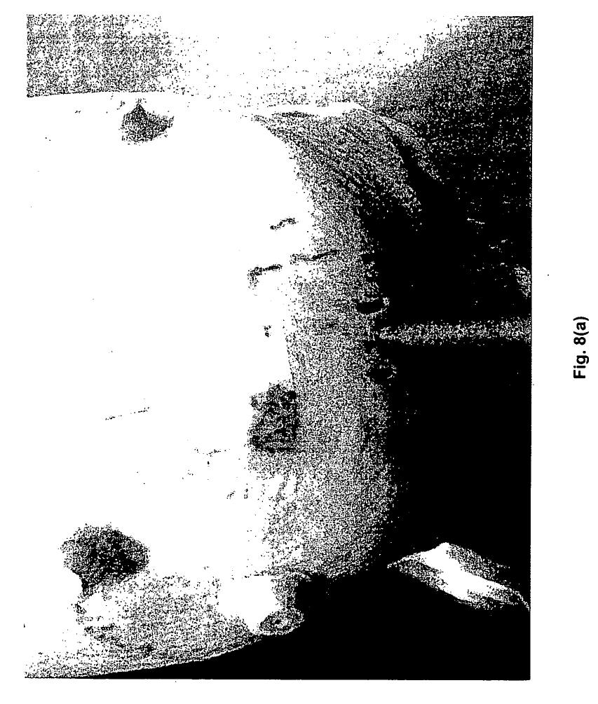

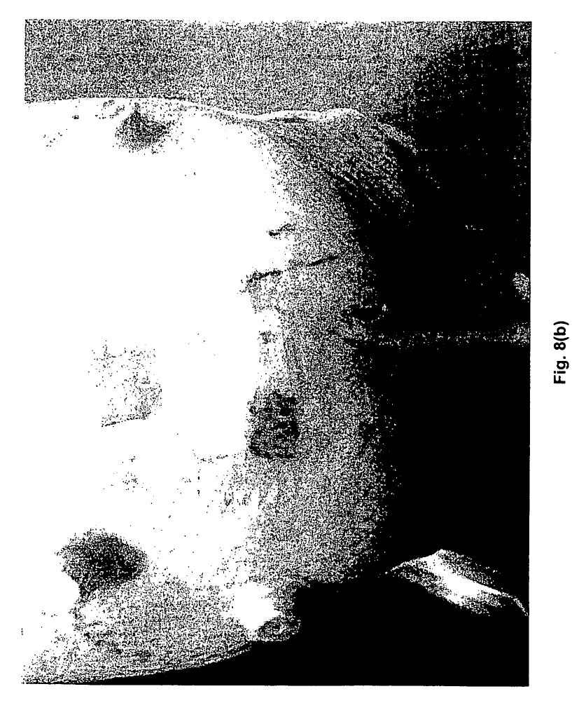

The resulting registration error shown in FIG. 7 suggests a very good registration result. The overlay of video and virtual images is quite good. This can be verified from inspecting an overlay image of the segmented phantom surface and the video image of the phantom, as is shown in FIGS. 8 (FIG. 8(a) is the original color image and FIG. 8(b) is an enhanced greyscale image).

FIG. 8 are a good example of an accurate overlay of virtual and real images. The video image of the background can be seen easily as there are no virtual objects there. The video image of the real skull can be seen (the small holes in front of the skull and the other fine features on the skull, such as set of the black squiggly lines near the center of the figure and the vertical black lines on the right border of the hole in the virtual skull, as well as the fiducials can be easily distinguished) although it is perfectly overlaid by the virtual image. There is a hole in the virtual image of the virtual skull (shown as surrounded by a zig-zag border) as that part of the virtual skull is not rendered because that part is nearer to the camera than a cutting plane defined to be at the probe tip's position and perpendicular to the camera. The virtual image of internal objects, here the virtual ball at the top left of the hole in the virtual skull which can not be seen in the video image, can be visualized.

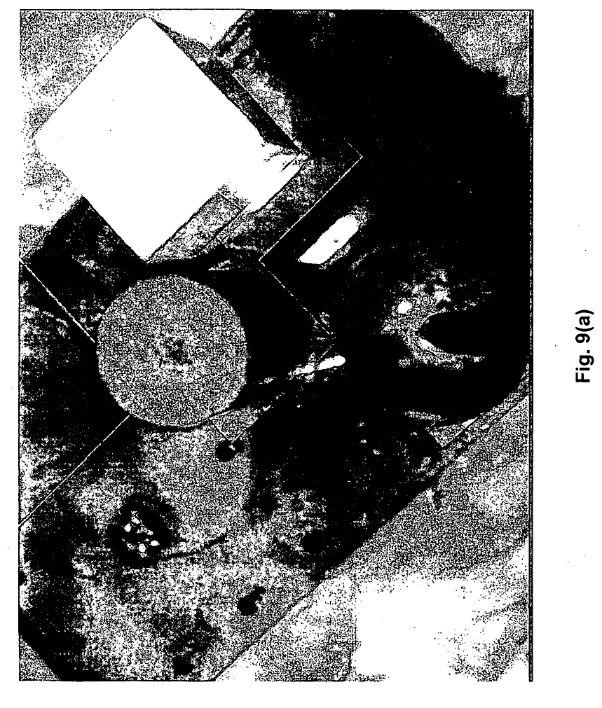

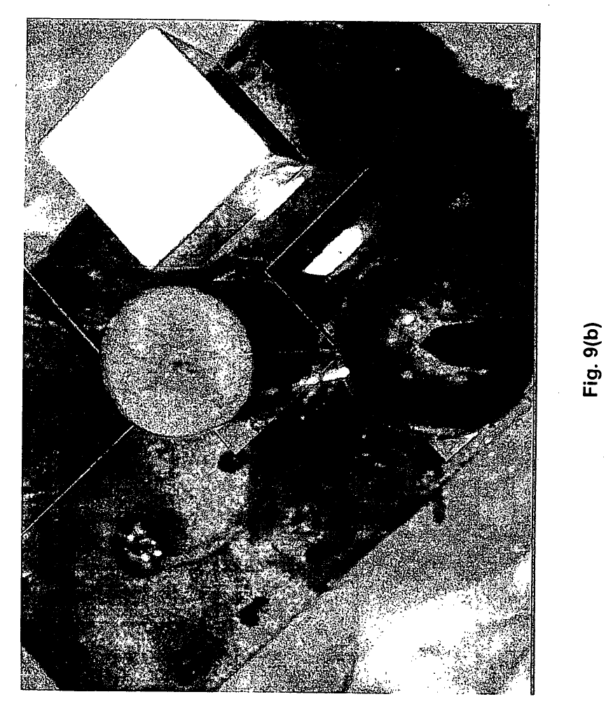

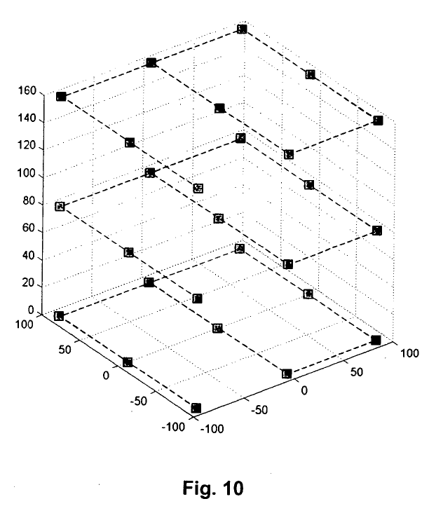

The registration error for target objects as shown in FIG. 9 was found as follows. The overlay error of the virtual and real target objects was easily assessed visually, as shown in FIG. 9 (FIG. 9(a) is the original color image and FIG. 9(b) is an enhanced greyscale image).

The registration error at a target object is normally hard to assess. However, because the overlay accuracy of the AR system had been evaluated using the methods of the present invention, and was proven to be much smaller than the overlay shown in FIG. 9 (see, for example, the extension of the video sphere above the red virtual sphere—in the exemplary test of FIG. 9 the virtual images of the target objects are colorized, and the real images were not), the registration error could be identified as the primary contribution to the overall error. Moreover, because it was known to a high degree of precision that the virtual geometric objects were precise models of their corresponding real objects it was concluded with some confidence that the overlay error in this exemplary test was caused mainly by registration error.

### EXAMPLE

The following example illustrates an exemplary evaluation of an AR system using methods and apparatus according to an exemplary embodiment of the present invention.

1. Accuracy Space

The accuracy space was defined as a pyramidal space associated with the camera. Its near plane to the viewpoint of the camera is 130 mm, the same as the probe tip. The depth of the pyramid is 170 mm. The height and width at the near plane are both 75 mm and at the far plane are both 174 mm, corresponding to a 512×512 pixels area in the image, as is illustrated in FIG. 5.

The overlay accuracy in the accuracy space was evaluated by eliminating the control points outside the accuracy space from the data set collected for the evaluation.

2. Equipment Used

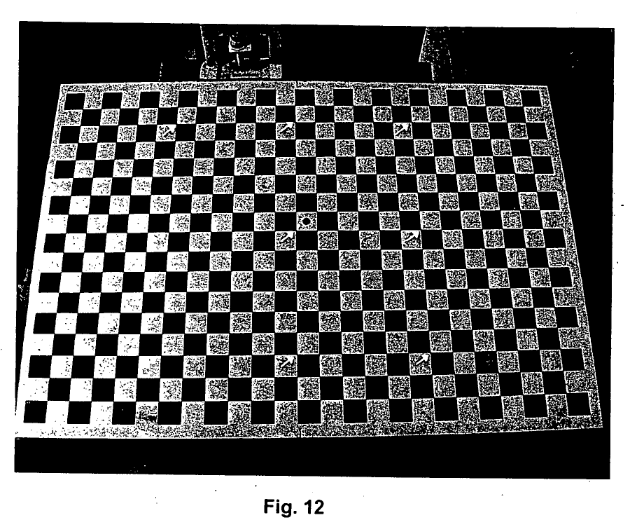

1. A motor driven linear stage which is made of a KS312-300 Suruga Z axis motorized stage, a DFC 1507P Oriental Stepper driver, a M1500, MicroE linear encoder and a MPC3024Z JAC motion control card. An adaptor plate was mounted on the stage with its surface vertical to the moving direction. The stage's travel distance is 300 mm, with an accuracy of 0.005 mm.
2. A planar test object which was made by gluing a printed chess square pattern on a planar glass plate. The test object is depicted in a close-up view in FIG. 12 and in the context of the entire test apparatus in FIG. 13. There were 17×25 squares in the pattern, with the size of each square being 15×15 mm. The corners of the chess squares were used as control points, as indicated by the arrows in FIG. 12.
3. Polaris hybrid tracking system.
4. A Traxtal TA-200 probe.
5. A DEX-Ray camera to be evaluated. As noted, DEX-Ray is an AR surgical navigation system developed by Volume Interactions Pte Ltd.
   3. Evaluation Method

An evaluation method according to an exemplary embodiment of the present invention was used to calculate the positional difference, or overlay error, of control points between their respective locations in the video and virtual images. The overlay error was reported in pixels as well as in millimeters (mm).

The linear stage was positioned at a proper position in the Polaris tracking space. The test object was placed on the adaptor plate. The calibrated DEX-Ray camera was held by a holder at a proper position above the test object. The complete apparatus is shown in FIG. 13. By moving the planar object with the linear stage, the control points were spread evenly across a volume, referred to as the measurement volume, and their 3D positions in the measurement volume were acquired. In the evaluation, it was made sure that the accuracy space of DEX-Ray™ was inside the measurement volume. A series of images of the calibration object at different moving steps was captured. By extracting the corners from these images, the positions of the control points in the real image were collected.

The corresponding 3D positions of the control points in a reference coordinate system defined on the test object were determined by the known corner positions on the test object and the distance moved. By detecting the 3D positions of some of these control points in the Polaris coordinate system, a transform matrix from the reference coordinate system to the Polaris coordinates was established by a registration process as described above. The reference frame's position and orientation on the probe were known through tracking. Thus, using the calibration data of the camera, a virtual image of the control points was generated and overlaid on the real images, in the same way as is done in the DEX-Ray system when virtual objects are combined with actual video images for surgical navigation purposes (in what has been sometimes referred to herein as an “application” use as opposed to an evaluation procedure as described herein).

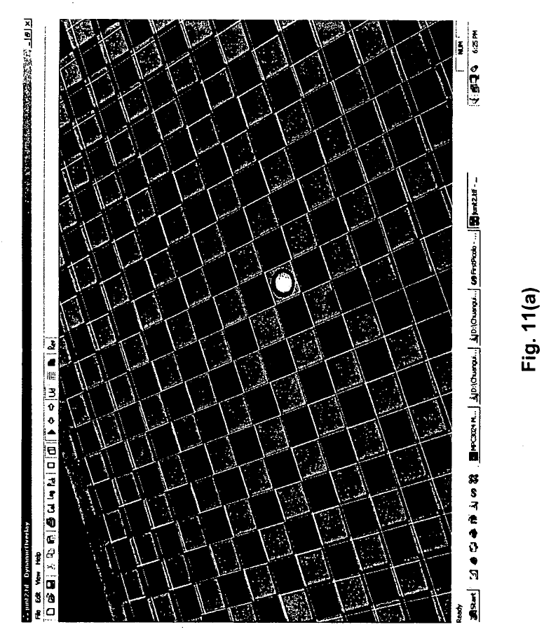

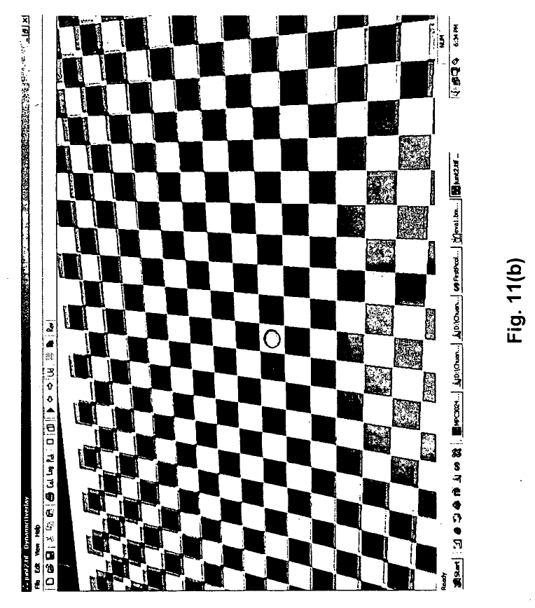

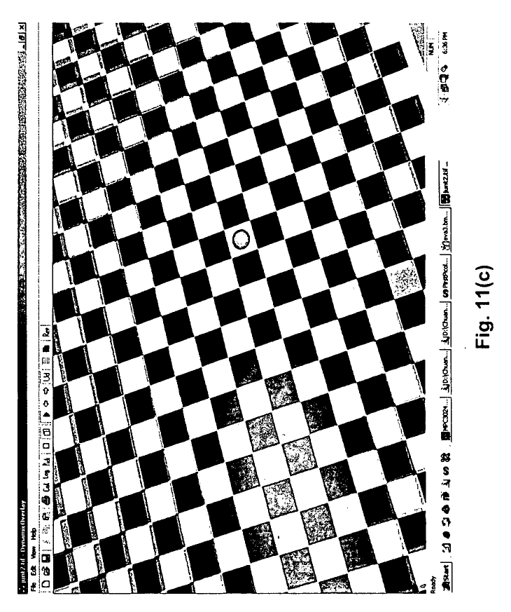

The above method can be used to evaluate thoroughly the overlay error at one or several camera positions. The overlay error at different camera rotations and positions in the Polaris tracking space can also be visualized by updating the overlay display in real time while moving the camera. Snapshots at different camera positions were used as another means to show the overlay accuracy. FIGS. 11 show the overlay at various exemplary camera positions.

4. Calibration Result

The DEX-Ray™ camera was calibrated using the same test object attached on the linear stage before the evaluation. The calibration results obtained were:
Camera Intrinsic Parameters:

$$Focal Length \text{:} fc = [ 883.67494 \qquad 887.94350 ] \pm [ 0.40902 \qquad 0.40903 ]$$

$$Principal point \text{:} cc = [ 396.62511 \qquad 266.49077 ] \pm [ 1.28467 \qquad 1.00112 ]$$

$$Skew \text{:} alpha\_c = [ 0.00000 ] \pm [ 0.00000 ]$$

$$\begin{matrix}Distortion \text{:} kc = [ - 0.43223 & 0.19703 & 0.00004 & - 0.00012 & 0.00000 ] \pm \\ [ 0.00458 & 0.01753 & 0.00020 & 0.00018 & 0.00000 ]\end{matrix}$$

Camera Extrinsic Parameters:

$$Orientation \text{:} omc = \begin{matrix}[ - 0.31080 & 0.27081 & 0.07464 ] \pm \\ [ 0.00113 & 0.0014 & 0.00031\end{matrix}$$

$$Position \text{:} Tc = \begin{matrix}[ - 86.32009 & - 24.31987 & 160.59892 ] \pm \\ [ 0.23802 & 0.187380 & 0.15752 ]\end{matrix}$$

Standard Pixel Error

$$err = [ 0.19089 \qquad 0.17146 ]$$

Camera to Marker Transform Matrix

$$Tcm = \begin{matrix}0.5190 & - 22.1562 & 117.3592\end{matrix}$$

$$Rcm = \begin{matrix}- 0.9684 & - 0.0039 & 0.2501 \\ 0.0338 & - 0.9929 & 0.1154 \\ 0.2479 & 0.1202 & 0.9615\end{matrix}$$

5. Evaluation Results

5.1 Registration of the Test Object

A Traxtal TA-200 probe was used to detect the coordinates of control points in the Polaris's coordinate system. The 3D locations of 9 control points, evenly spread on the test object with a distance of 90 mm, were picked up. The test object was moved 80 mm and 160 mm downwards, and the same process was repeated. So altogether there were 27 points used to determine the pose of the test object to Polaris as shown in FIG. 10. The transform matrix from the evaluation object to Polaris was calculated as:

$$Tot = \begin{matrix}93.336 & 31.891 & - 1872.9\end{matrix}$$

$$Rot = \begin{matrix}- 0.88879 & - 0.25424 & 0.38135 \\ - 0.45554 & 0.39842 & - 0.79608 \\ 0.050458 & - 0.88126 & - 0.46992\end{matrix}$$

The exemplary registration algorithm used is in Matlab as follows:
X=Coordinates of control points in Test Object coordinate system
Y=Coordinates of control points in Polaris coordinate system
Ymean=mean(Y)′;
Xmean=mean(X)′;
K=(Y′−Ymean*ones(1, length(Y)))*(X′−Xmean*ones(1,length(X)))′;
[U,S,V]=svd(K);
D=eye(3,3); D(3,3) =det(U*V′);
R=U*D*V′;
T=Ymean−R*Xmean;
Rot=R′; Tot=T′;
%%%Registration error
   Registration Error=(Y-ones(length(X),1)*Tot)*inv(Rot)-X;

X specifies the coordinates of the 27 control points in the test object coordinate system. Y specifies the coordinates of the 27 control points in Polaris' coordinate system, as shown in Table A below.

TABLE A

| X | | | Y | | | Registration Error | | |
|---|---|---|---|---|---|---|---|---|
| 0 | 90 | 0 | 52.724 | 67.681 | −1943.8 | −0.044264 | −0.72786 | −0.22387 |
| 0 | 0 | 0 | 93.377 | 31.736 | −1872.9 | 0.019906 | −0.054977 | 0.13114 |
| 0 | −90 | 0 | 134.51 | −3.896 | −1801.4 | −0.22025 | 0.091169 | −0.019623 |
| 90 | −90 | 0 | 54.305 | −26.971 | −1767.2 | −0.043994 | 0.25427 | 0.22521 |
| 90 | 0 | 0 | 13.364 | 9.032 | −1838.9 | −0.14493 | 0.31594 | 0.14737 |
| 90 | 90 | 0 | −27.679 | 44.905 | −1910.1 | 0.058586 | −0.0050323 | −0.043916 |
| −90 | 90 | 0 | 132.37 | 90.779 | −1978.8 | −0.040712 | 0.029275 | −0.13028 |
| −90 | 0 | 0 | 173.32 | 54.681 | −1907.4 | −0.024553 | 0.14554 | 0.16035 |
| −90 | −90 | 0 | 214.25 | 18.908 | −1835.7 | 0.012441 | 0.21242 | 0.053781 |
| 0 | 90 | 80 | 56.406 | −2.7 | −1982.3 | −0.10223 | 0.16771 | 0.073327 |
| 0 | 0 | 80 | 97.479 | −38.499 | −1910.4 | −0.076278 | −0.069355 | −0.13808 |
| 0 | −90 | 80 | 138.39 | −74.314 | −1839 | −0.10134 | 0.18342 | −0.094966 |
| 90 | −90 | 80 | 58.325 | −97.196 | −1804.9 | −0.11446 | 0.37436 | −0.0019349 |
| 90 | 0 | 80 | 17.4 | −61.509 | −1876.2 | −0.013908 | 0.020188 | 0.032556 |
| 90 | 90 | 80 | −23.637 | −25.805 | −1947.7 | 0.10865 | −0.12336 | 0.13671 |
| −90 | 90 | 80 | 136.41 | 20.256 | −2016.4 | −0.035532 | 0.00074754 | −0.10829 |
| −90 | 0 | 80 | 177.29 | −15.721 | −1944.6 | 0.15319 | −0.11817 | −0.11119 |
| −90 | −90 | 80 | 218.34 | −51.686 | −1873.1 | 0.085047 | −0.076872 | 0.018895 |
| 0 | 90 | 160 | 60.337 | −73.316 | −2019.5 | 0.19152 | −0.21518 | −0.042746 |
| 0 | 0 | 160 | 101.44 | −109.28 | −1947.8 | 0.11251 | −0.28752 | 0.039059 |
| 0 | −90 | 160 | 142.46 | −144.75 | −1876.6 | −0.18026 | 0.22463 | −0.11249 |
| 90 | −90 | 160 | 62.452 | −167.96 | −1842.3 | −0.05999 | 0.057679 | 0.15009 |
| 90 | 0 | 160 | 21.461 | −132.01 | −1913.8 | −0.062087 | 0.035828 | 0.068357 |
| 90 | 90 | 160 | −19.564 | −96.075 | −1985.2 | 0.042176 | −0.12814 | −0.097016 |
| −90 | 90 | 160 | 140.27 | −50.351 | −2053.8 | 0.22446 | −0.14881 | −0.11926 |
| −90 | 0 | 160 | 181.34 | −86.321 | −1982.2 | 0.14631 | −0.15297 | −0.0011792 |
| −90 | −90 | 160 | 222.3 | −122.15 | −1910.7 | 0.10999 | −0.0049041 | 0.0080165 |

5.2 Tracking Data

The camera was held at a proper position above the test object. It was kept still throughout the entire evaluation process. The Polaris sensor was also kept still during the evaluation. The reference frame on the DEX-Ray™ probe's position and orientation to Polaris were:

$$Trt = \begin{matrix}180.07 & 269.53 & - 1829.5\end{matrix}$$

$$Rrt = \begin{matrix}0.89944 & - 0.40944 & - 0.15159 \\ 0.09884 & - 0.14717 & 0.98396 \\ - 0.42527 & - 0.90017 & - 0.091922\end{matrix}$$

5.3 Video Image

The test object was moved close to the camera after registration. The distance which it was moved was automatically detected by the computer through the feedback of the encoder. A video image was captured and stored. Than the test object was moved down 20 mm and stopped, and another video image was captured and stored. This process was continued until the object was out of the measurement volume. In this evaluation, the total distance moved was 160 mm. Eight video images were taken altogether. (An image at 160 mm was out of the measurement volume and thus was not used.)

5.4 Evaluation Results

Using the calibrated data, the registration data test object, the tracking data of the reference frame, and the moved distance of the test object, the control points' locations to the camera were be determined and virtual images of the control points at each movement step were generated as described above.

The positional difference between the control points in the video image at each movement step and the corresponding control points in the virtual image at that movement step were be calculated. The overlay accuracy was calculated using the methods described above.

The overlay accuracy across the whole working space of the DEX-Ray system was evaluated. The maximum, mean and RMS errors at the probe position evaluated were 2.24312, 0.91301, and 0.34665 in pixels. Mapping to objective space, the corresponding values were 0.36267, 0.21581, and 0.05095 in mm.

It is noted that the above-described process can be used to evaluate the overlay accuracy at various camera positions and orientations. It is also possible to visualize the overlay accuracy dynamically, in a similar way as in a real application. Some snapshots of the overlay display at different camera positions are shown in FIGS. 11. Although the evaluation result was obtained at only one camera position, these snapshots indicate that it is true at normal conditions.

References

The following references provide background and context to the various exmeplary embodiemtns of the present invention described herein.
[1] P J. Edwards, etc, Design and Evaluation of a System for Microscope-Assisted Guided Interventions (MAGI), IEEE Transactions on Medical Imaging, vol. 19, No. 11, November 2000.
[2] W. Birkfeller, etc, Current status of the Varioscope A R, a head-mounted operating microscope for computer-aided surgery, IEEE and ACM International Symposium on Augmented Reality (ISAR′01) , Oct. 29-30, 2001, New York, New York.

[3] W. Grimson, etc, An Automatic Registration Method for Frameless Stereotaxy, Image Guided Surgery, and Enhanced Reality Visualization, Transactions on Medical Imaging, vol. 15, No. 2, April 1996 .

[4] William Hoff, Tyrone Vincent, Analysis of Head Pose Accuracy in Augmented Reality. IEEE Transactions on Visualization and Computer Graphics, vol. 6, No. 4, October-December 2000.

[5] A. P. King, etc, An Analysis of calibration and Registration Errors in an Augmented Reality System for Microscope-Assisted Guided Interventions, Proc. Medical Image Understanding and Analysis 1999.

The foregoing description merely illustrates the principles of the present invention and it will thus be appreciated that those skilled in the art will be able to devise numerous alternative arrangements which, although not explicitly described herein, embody the principles of the invention and are within its spirit and scope.

1. A method of measuring overlay error in augmented reality systems, comprising:
providing a test object;
registering the test object;
capturing images of one or more reference points on the test object at various positions within a defined workspace;
extracting positions of reference points on the test object from the captured images;
calculating re-projected positions of the reference points; and
calculating the differences between the extracted and re-projected reference points.

2. The method of claim 1, wherein the test object is one of planar, bi-planar, volumetric or comprising a single point.

3. The method of claim 1, wherein the test object is moved within the defined workspace by precisely known increments to acquire multiple positions for each of the reference points.

4. The method of claim 1, wherein the test object is precisely manufactured or measured such that the distances between successive reference points are substantially equal to within known tolerances.

5. The method of claim 1, wherein the test object has one or more pivots, and wherein the distances from said pivots to the reference points are precisely known to within defined tolerances.

6. The method of claim 3, wherein at least three positions for each reference point are acquired.

7. The method of claim 1, wherein calculation of the differences between the extracted and re-projected reference points is as to each reference point and includes calculation of one or more of a minimum, maximum, mean and standard deviation over all reference points within the defined workspace.

8. The method of claim 1, further comprising determining whether given the overall differences between all of the extracted and re-projected reference points the augmented reality system meets a given standard.

9. The method of claim 1, further comprising using the overall differences between all of the extracted and re-projected reference points as a baseline against which to measure other sources of overlay error.

10. The method of claim 9, wherein said other sources of overlay error include registration error.

11. A method of measuring overlay error in augmented reality systems, comprising:
providing a real test object;
generating a virtual test object;
registering the real test object to the virtual test object;
capturing images of one or more reference points on the test object and generating virtual images of corresponding points on the virtual test
object at various positions within a defined workspace;
extracting positions of reference points on the real test object from the captured images;
extracting corresponding positions of said reference points on the virtual test object from the virtual images; and
calculating the positional differences between the real and virtual reference points.

12. The method of claim 1, wherein the test object is one of planar, bi-planar, volumetric or comprising a single point.

13. The method of claim 11, wherein the test object is moved within the defined workspace by precisely known increments to acquire multiple positions for each of the reference points.

14. The method of claim 11, wherein the test object is precisely manufactured or measured such that the distances between successive reference points are substantially equal to within known tolerances.

15. The method of claim 11, wherein the test object has one or more pivots, and wherein the distances from said pivots to the reference points are precisely known to within defined tolerances.

16. The method of claim 13, wherein at least three positions for each reference point are acquired.

17. The method of claim 11, wherein calculation of the differences between the extracted and re-projected reference points is as to each reference point and includes calculation of one or more of a minimum, maximum, mean and standard deviation over all reference points within the defined workspace.

18. A system for measuring overlay error in an augmented reality system, comprising:
a test object with one or more defined reference points;
a tracking device;
a data processor;
a camera or imaging device used in the AR system,
wherein the test object and camera can each be tracked in a tracking space of the tracking system, and wherein in operation the camera or imaging system generates one or more images of the test object and the data processor generates an equal number of virtual images of a corresponding virtual test object at various positions in a defined workspace and locational differences between corresponding reference points are calculated.

19. The system of claim 18, wherein the test object is one of panar, bi-planar, volumetric or comprising a single point.

20. The system of claim 18, wherein in operation the test object is moved within the defined workspace by precisely known increments to acquire multiple positions for each of the reference points.

21. The system of claim 18, wherein the test object is precisely manufactured or measured such that the distances between successive reference points are substantially equal to within known tolerances.

22. The system of claim 18, wherein the test object has one or more pivots, and wherein the distances from said pivots to the reference points are precisely known to within defined tolerances.

23. The system of claim 18, wherein in operation the camera or imaging device is held fixed at a defined position relative to the tracking device while the one or more images are being generated.

24. The system of claim 18, wherein the test object has a single reference point and is stepped throughout a defined workspace via a CMM.

25. The method of claim 1, wherein the defined workspace is a space associated with the camera or imaging system.

26. The system of claim 20, wherein the defined workspace is a space associated with the camera or imaging system.
